(19) United States
(12) Patent Application Publication
(10) Pub. No.: US 2012/0257030 A1
(43) Pub. Date: Oct. 11, 2012

(54) ENDOSCOPE APPARATUS AND IMAGE ACQUISITION METHOD OF THE ENDOSCOPE APPARATUS

(75) Inventors: Jae-guyn LIM, Seongnam-si (KR); Won-hee Choe, Seoul (KR); Seong-deok Lee, Seongnam-si (KR)

(73) Assignee: SAMSUNG ELECTRONICS CO., LTD., Suwon-si (KR)

(21) Appl. No.: 13/225,668

(22) Filed: Sep. 6, 2011

(30) Foreign Application Priority Data
Apr. 8, 2011 (KR) 10-2011-0032723

(51) Int. Cl. H04N 7/18

(52) U.S. Cl. 348/70; 348/E07.085

(57) ABSTRACT

An endoscope apparatus is provided. The endoscope apparatus includes an illumination unit configured to selectively provide light of different wavelength bands and white light, a sensing unit configured to generate an image signal by receiving light of a near infrared wavelength band and light of a visible light band, and an image processing unit configured to generate a color image and a plurality of narrow band images from image signals of different wavelength bands generated by the sensing unit. The sensing unit includes a plurality of photodetection pixels, each photodetection pixel comprising an infrared sub-pixel for sensing near infrared light, a red sub-pixel for sensing red light, a blue sub-pixel for sensing blue light, and a green sub-pixel for sensing green light.

### CROSS-REFERENCE TO RELATED APPLICATIONS

This application claims the benefit of Korean Patent Application No. 10-2011-0032723, filed on Apr. 8, 2011 in the Korean Intellectual Property Office, the entire disclosure of which is incorporated herein by reference for all purposes.

### BACKGROUND

1. Field

The following description relates to an endoscope apparatus and image acquisition method of the endoscope apparatus.

2. Description of the Related Art

An endoscope is a medical tool that is used by being inserted in a body, for example, a human body, in order to enable direct observation of an organ or a cavity, for example, a body cavity, in which a lesion may not be observed without an operation or incision. An initial endoscope has a narrow, long insertion portion, which may be inserted into a cavity to facilitate observation of an organ in the cavity. As image processing technology develops, a black and white camera is used to capture an image for each part in the cavity and thus a lesion in each part may be examined through the captured images. Then, for example, the simple black and white camera has been replaced by a high-resolution color imaging device so that a lesion may be observed in more detail. In another example, a chromoendoscope may be used. The chromoendoscope captures an image after dyeing a surface of the cavity with a particular pigment according to the type of a lesion to be identified.

Recently, a narrow band imaging (NBI) endoscope has been suggested to provide an improved lesion identification power. The NBI endoscope functions based on the principle that a depth of light that penetrates tissue varies according to the wavelength of the light. For example, the NBI endoscope captures an image of each part in the cavity using a blue, green, or red light of a narrow wavelength band instead of using a general white light of a wide wavelength band. Accordingly, an image of a surface, a middle part or a deep part of a mucous membrane in the cavity may be obtained according to the wavelength of the light used. Thus, a lesion may be easily identified from a difference between the obtained images. The NBI endoscope may be used to determine, for example, esophageal angiodysplasia or stomach cancer in its early stage where the esophageal angiodysplasia or stomach cancer has not yet protruded, tumor lesions of a large intestine, and loss of normal vessels.

Currently, the NBI endoscope sequentially or selectively illuminates an interested part within the cavity with visible light of blue, green, and red narrow bands to obtain various images captured at the different bands of visible light. The NBI endoscope using visible light is capable of identifying capillary vessels from other areas at a mucosal layer depth of about 200 to 300 μm.

### SUMMARY

According to an aspect, an endoscope apparatus is provided. The endoscope apparatus includes an illumination unit configured to selectively provide light of different wavelength bands and white light, a sensing unit configured to generate an image signal by receiving light of a near infrared wavelength band and light of a visible light band, and an image processing unit configured to generate a color image and a plurality of narrow band images from image signals of different wavelength bands generated by the sensing unit. The sensing unit includes a plurality of photodetection pixels, each photodetection pixel comprising an infrared sub-pixel for sensing near infrared light, a red sub-pixel for sensing red light, a blue sub-pixel for sensing blue light, and a green sub-pixel for sensing green light.

The illumination unit may include a white light source for emitting light of a near infrared ray band and white light of a visible light band, and a filter member comprising a visible light filter that allows visible light to pass through and a narrow band filter that allows light of a plurality of different wavelength bands to pass through.

The narrow band filter may allow blue light, green light, and near infrared light to pass through, and light that is emitted from the white light source and passes through the narrow band filter becomes light in which blue, green, and near infrared light are mixed.

The filter member may be disposed on a light path in front of the white light source and is moved or rotated such that light emitted from the white light source is selectively passed through the visible light filter or the narrow band filter.

The illumination unit may include a white light source for emitting white visible light of a wide band, a blue light source for emitting narrow band blue light, a green light source for emitting narrow band green light, and a near infrared ray light source for emitting narrow band near infrared light.

The endoscope apparatus may operate in a white light mode that provides white visible light to a cavity or a narrow band mode that provides light of a plurality of narrow bands to the cavity. In the white light mode, the white light source is turned on and the blue, green, and near infrared light sources may be turned off, and in the narrow band mode, the white light source may be turned off, and the blue, green, and near infrared light sources may be turned on.

The illumination unit may include a blue light source for emitting narrow band blue light, a green light source for emitting narrow band green light, a red light source for emitting narrow band red light, and a near infrared ray light source for emitting narrow band near infrared light.

The endoscope apparatus may operate in a white light mode that provides white visible light to a cavity or a narrow band mode that provides light of a plurality of narrow bands to the cavity. In the white light mode, the blue, green, and red light sources may be turned on and the near infrared light source may be turned off, and in the narrow band mode, the red light source may be turned off, and the blue, green, and near infrared light sources may be turned on.

The endoscope apparatus may operate in a white light mode that provides white visible light to a cavity or a narrow band mode that provides light of a plurality of narrow bands to a cavity, and in the white light mode, the image processing unit may generate a color image based on image signals transmitted from the sensing unit.

In the narrow band mode, the image processing unit may generate a narrow band blue image, a narrow band green image, a narrow band near infrared image based on the image signals transmitted from the sensing unit.

The image processing unit may generate additional narrow band images by fusing any two of or all of the blue, green, and near infrared narrow band images or fusing a color image obtained in the white light mode and one of the blue, green, and near infrared narrow band images obtained in the narrow band mode.

The image processing unit may compensate for discoloration and a decrease in resolution generated in the sensing unit by using an interpolation method.

Each of the photodetection pixels may include a substrate, a plurality of photosensitive layers arranged on the substrate, and a blue color filter, a green color filter, a red color filter, and a near infrared ray filter disposed on corresponding photosensitive layers.

The image processing unit may include values that are measured with respect to ratios at which near infrared rays are sensed at the red sub-pixel, the blue sub-pixel, and the green sub-pixel with respect to the near infrared sub-pixel.

The image processing unit may calculate a contribution ratio of the near infrared rays among the amount of light measured at each of the red, blue, and green sub-pixels based on the values measured in advance, and corrects red, blue, and green color information in the red, blue, and green sub-pixels based on a calculation result.

Each of the photodetection pixels may further include an infrared ray cut-off filter that is disposed on the blue color filter, the green color filter, and the red color filter.

The visible light filter may filter light other than the visible light.

The narrow band filter may filter light other than the blue, green and near infrared bands.

In another aspect, a method of obtaining an image of an endoscope apparatus is provided. The method includes illuminating a cavity by selectively providing light of a plurality of narrow bands of different wavelength bands including a near infrared band and white visible light to the cavity, generating an image signal with respect to the plurality of different wavelength bands by receiving light reflected by the cavity, generating a color image and a plurality of narrow band images from image signals with respect to the plurality of different wavelength bands, and generating additional narrow band images by fusing the color image and the plurality of narrow band images.

The illuminating may be performed in a white light mode in which white visible light is provided to the cavity and a narrow band mode in which light of a plurality of narrow band wavelengths is provided to the cavity.

The light of a plurality of narrow band wavelengths may include narrow band blue light, narrow band green light, and narrow band near infrared light.

The generating a color image and a plurality of narrow band images may include generating a narrow band blue image, a narrow band green image, and a narrow band near infrared image based on image signals with respect to the narrow band blue light, the narrow band green light, and the narrow band near infrared light.

The generating a color image and a plurality of narrow band images may include generating a color image based on image signals with respect to the white visible light.

The generating additional narrow band images may include generating additional narrow band images by fusing any two narrow band images among a narrow band blue image, a narrow band green image, and a narrow band near infrared image or all the narrow band images or fusing the color image with the narrow band blue image, the green band narrow image, and the narrow band near infrared image.

The generating an image signal may include compensating for discoloration and a decrease in a resolution in a sensing unit that receives light reflected by the cavity, by using an interpolation method.

The sensing unit may include a plurality of photodetection pixels, each photodetection pixel comprising an infrared sub-pixel for sensing near infrared light, a red sub-pixel for sensing red light, a blue sub-pixel for sensing blue light, and a green sub-pixel for sensing green light.

The generating an image signal may include calculating a contribution ratio of the near infrared rays among the light amount measured at each of the red, blue, and green sub-pixels based on values measured with respect to ratios at which near infrared rays are sensed by the red sub-pixel, the blue sub-pixel, and the green sub-pixel with respect to the near infrared sub-pixel, and correcting red, blue, and green color information of the red, blue, and green sub-pixels based on a calculation result.

In yet another aspect, an endoscope apparatus is provided. The endoscope apparatus includes an illumination unit configured to emit light including red, green and blue bands and near infrared band, a sensing unit configured to receive the light via at least one photodetection pixel, each including one sub-pixel corresponding to each one of the bands of the light, and an image processing unit configured to generate an image based on the received light.

Other features and aspects may be apparent from the following detailed description, the drawings, and the claims.

### BRIEF DESCRIPTION OF THE DRAWINGS

FIG. 1 is a diagram illustrating an example of an endoscope apparatus;

FIG. 2 is a diagram illustrating an example of an illumination unit illustrated in FIG. 1;

FIG. 3 is a graph illustrating pass bands of a narrow filter used in the illumination unit of FIG. 2;

FIG. 4 is a view illustrating an example of a photodetection pixel of a sensing unit illustrated in FIG. 1;

FIG. 5A illustrates a cross-sectional view of the example of the photodetection pixel illustrated in FIG. 4 taken along a line A-A′;

FIG. 5B illustrates a cross-sectional view of the example of the photodetection pixel illustrated in FIG. 4 taken along a line B-B′;

FIG. 6 illustrates another example of the illumination unit of FIG. 1;

FIG. 7 illustrates another example of the illumination unit of FIG. 1;

FIG. 8 illustrates a view of an example of a photodetection pixel of the sensing unit illustrated in FIG. 1;

FIG. 9A illustrates a cross-sectional view of the example of the photodetection pixel illustrated in FIG. 8 taken along a line A-A′; and

FIG. 9B illustrates a cross-sectional view of the example of the photodetection pixel illustrated in FIG. 8 taken along a line B-B′.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be suggested to those of ordinary skill in the art. Also, descriptions of well-known functions and constructions may be omitted for increased clarity and conciseness.

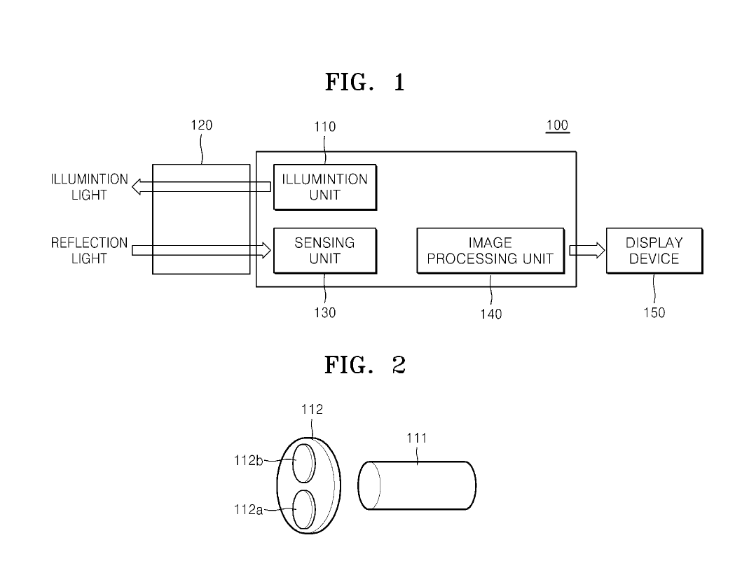

FIG. 1 is a diagram illustrating an example of an endoscope apparatus 100. Referring to FIG. 1, the endoscope apparatus 100 may include an illumination unit 110 that may selectively provide light having a plurality of relatively narrow wavelength bands and white light of a relatively wide band, a sensing unit 130 that receives light of a relatively wide band, including an infrared wavelength band and a visible light band, to generate an image signal, and an image processing unit 140 that may generate new images with improved lesion identification power by fusing the image signal of different wavelength bands generated by the sensing unit 130. The endoscope apparatus 100 may further include a light transmission member 120 that transmits light generated by the illumination unit 110 to a desired portion of a cavity. As another aspect, in response to the illumination unit 110 and the sensing unit 130 being directly inserted into the cavity, the light transmission member 120 may not be employed. Also, the endoscope apparatus 100 may further include a display device 150 that displays an image generated by the sensing unit 130 and the image processing unit 140.

FIG. 2 is a diagram illustrating an example of the illumination unit 110 of FIG. 1. Referring to FIG. 2, the illumination unit 110 may include a white light source 111 which may emit white light of a relatively wide band. The white light emitting the relatively wide band may include a near infrared band. For example, a xenon lamp may be used as the white light source 111. The illumination unit 110 may also include a filter member 112 including a visible light filter 112a that allows visible light to pass through and a narrow band filter 112b that allows multiple narrow band light of a plurality of different wavelength bands to pass through. For example, the visible light filter 112a may allow visible rays having a wavelength in a range from about 400 to about 700 nm to pass through, and may filter non-visible rays. Accordingly, light that is emitted from the white light source 111 and passes through the visible light filter 112a becomes pure white visible light.

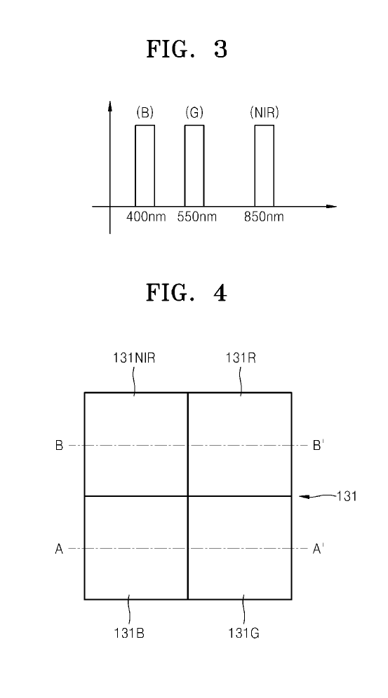

The narrow band filter 112b may be configured to allow to pass through, for example, narrow band light such as blue, green, and near infrared bands, and filtering out wider band light. FIG. 3 is a graph illustrating pass bands of the narrow band filter 112b. Referring to FIG. 3, the narrow band filter 112b may allow to pass through, for example, light of a blue wavelength band of about 400 nm to about 450 nm, light of a green wavelength band of about 500 to 560 nm, and light of a near infrared wavelength band of about 800 to 860 nm. Accordingly, light that is emitted from the white light source 111 and passes through the narrow band filter 112b becomes narrow band light in which blue narrow band light, green narrow band light, and near infrared narrow band light may be mixed. As another aspect, the above-described figures of the pass bands are an example, and it is understood that the specific range of pass bands of the narrow band filter 112b that vary are within the scope of the teachings herein.

The filter member 112 may be disposed on a light path in front of the white light source 111 such that light emitted from the white light source 111 selectively passes through the visible light filter 112a or the narrow band filter 112b. For example, the filter member 112 may be manufactured in the form of a rotary filter wheel. In this case, the visible light filter 112a and the narrow band filter 112b may be respectively disposed as a first half circle area and a second half circle area of the filter member 112 having a thin disc shape. According to an example, the filter member 112 may be rotated or moved in order to selectively locate the visible light filter 112a and the narrow band filter 112b on the light path.

The light transmission member 120 is a narrow, long insertion portion configured to be inserted into a cavity. The light transmission member 120 provides the light emitted by the illumination unit 110 into the cavity to illuminate a particular portion of the cavity. Also, the light transmission member 120 transmits light reflected from an illuminated portion of the cavity to the sensing unit 130. To this end, the light transmission member 120 may include, for example, a plurality of optical fibre bundles (not shown). Since the light transmission member 120 may be the same as one used in a general endoscope apparatus, a further description of the light transmission member 120 is omitted herein for conciseness.

The sensing unit 130 senses the reflected light transmitted by the light transmission member 120 and forms an image signal corresponding to the illuminated portion of the cavity. To this end, the sensing unit 130 may include a plurality of photodetection pixels 131 arranged in a 2D array. FIG. 4 illustrates an example of a photodetection pixel 131 of the sensing unit 130 of FIG. 1. Referring to FIG. 4, the photodetection pixel 131 of the sensing unit 130 may include four sub-pixels. For example, each photodetection pixel 131 of the sensing unit 130 may include a near infrared sub-pixel 131NIR for sensing near infrared light, a red sub-pixel 131R for sensing red light, a blue sub-pixel 131B for sensing blue light, and a green sub-pixel 131G for sensing green light. Accordingly, the sensing unit 130 may sense near infrared rays by using the near infrared sub-pixel 131NIR. As another example, a CMOS image sensor or a CCD image sensor that is generally used may be used as a photodetection device of the sub-pixels, namely, the near infrared, red, blue, and green sub-pixels 131NIR, 131R, 131G, and 131B. As another example, an image sensor having a back-side illumination (BSI) structure may be used in order to increase sensitivity with respect to narrow band light.

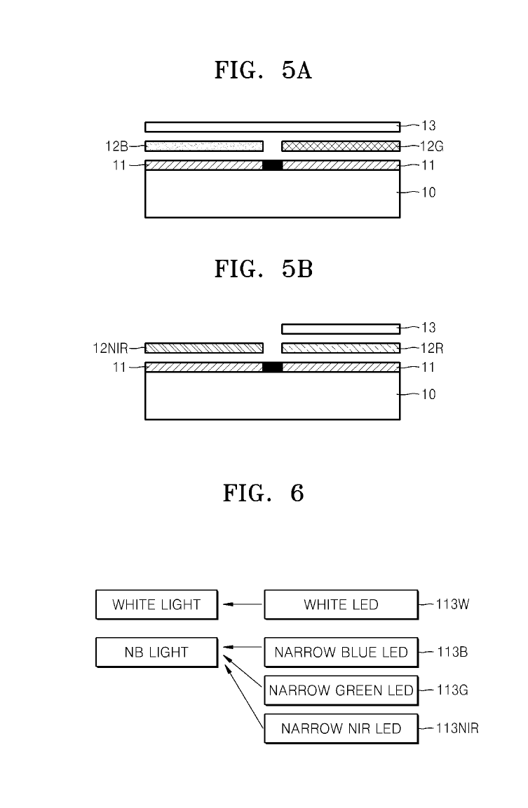

To prevent near infrared rays from affecting the red sub-pixel 131R, the blue sub-pixel 131B, and the green sub-pixel 131G, an infrared (IR) cut-off filter 13 may be disposed on the red sub-pixel 131R, the blue sub-pixel 131B, and the green sub-pixel 131G. FIGS. 5A and 5B illustrate cross-sectional views of an example of the photodetection pixel 131. FIG. 5A illustrates a cross-sectional view of the example of the photodetection pixel 131 taken along a line A-A′, and FIG. 5B illustrates a cross-sectional view of the example of the photodetection pixel 131 taken along a line B-B′.

Referring to FIG. 5A, a plurality of photosensitive layers 11 including photosensitive devices and wirings are disposed on the substrate 10, and a blue color filter 12B and a green color filter 12G are disposed on corresponding photosensitive layers 11. An IR cut-off filter 13 is disposed on the blue color filter 12B and the green color filter 12G. Referring to FIG. 5B, the plurality of photosensitive layers 11 are disposed on the substrate 10, and a near infrared filter 12NIR and a red color filter 12R are disposed on corresponding photosensitive layers 11. As another aspect, the IR cut-off filter 13 is disposed on the red color filter 12R, and the IR cut-off filter 13 is not disposed on the near infrared filter 12NIR. Accordingly, near infrared rays have substantially no effect on the red sub-pixel 131R, the blue sub-pixel 131B, and the green sub-pixel 131G and may be sensed by the near infrared sub-pixel 131NIR.

The image processing unit 140 may process an image signal obtained from the sensing unit 130 to generate a narrow band image having a high lesion contrast. Hereinafter, the operation of the endoscope apparatus 100 will be described based on the operation of the image processing unit 140.

In a white light mode of an initial operation of the endoscope apparatus 100, the visible light filter 112a of the filter member 112 may be located on a light path. Among light of various wavelength bands emitted from the white light source 111, white light having a visible light component is provided to a selected portion of a cavity via the light transmission member 120. The light reflected by the selected portion of the cavity is illuminated by the provided white visible light. The reflected light is transmitted to the sensing unit 130 via the light transmission member 120. Each of the sub-pixels, namely, the near infrared, red, blue, and green sub-pixels 131NIR, 131R, 131G, and 131B, of the sensing unit 30 may receive the visible light reflected by the selected portion of the cavity, and based on the reflected light, generate at least one color image signal. Since white visible light includes almost no infrared component, the at least one image signal will be generated substantially based on the red, green, and blue sub-pixels 131R, 131G, and 131B.

Unlike a color pixel of a general Bayer pattern in which one blue sub-pixel, one red sub-pixel, and two green sub-pixels are included in a single pixel, a single pixel includes one green sub-pixel. Accordingly, the image processing unit 140 may compensate for discoloration and a decrease in resolution using at least one interpolation method. For example, the image processing unit 140 may add a weight to an image signal from the green sub-pixel 131G, and may apply an interpolation method such as, for example, bilinear interpolation, bicubic interpolation, or median interpolation to image signals from all the sub-pixels.

The image processing unit 140 may generate a color image based on the compensated image signals. A color image of an inner portion of the cavity generated by the image processing unit 140 may be displayed on the display device 150, for example, in real-time, or after a time delay. The user may view the color image displayed on the display device 150 to check for any suspicious lesion portion. If a suspicious lesion portion is not found, observation may continue by selecting other portions of the cavity.

As another aspect, in response to a suspicious lesion portion being found in a particular portion of the cavity, the endoscope apparatus 100 may be switched to a narrow band mode for generating a narrow band image to more accurately identify a lesion. During the switching, for example, the filter member 112 may be moved or rotated to replace the visible light filter 112a, which is currently in the light path, with the narrow band filter 112b. Then, among light of various wavelength bands emitted from the white light source 111, light of a blue band, a green band, and a near infrared band is provided to the selected portion of the cavity via the light transmission member 120. Light reflected by the selected portion of the cavity is transmitted to the sensing unit 130 via the light transmission member 120. Each of the sub-pixels, namely, the near infrared, red, blue, and green sub-pixels 131NIR, 131R, 131G, and 131B, of the sensing unit 130 may generate an image signal with respect to each of the wavelength bands by receiving the light reflected by the selected portion of the cavity. The image processing unit 140 may apply at least one of the interpolation methods described above with respect to an image signal from each of the sub-pixels, namely, the near infrared, red, blue, and green sub-pixels 131NIR, 131R, 131G, and 131B to obtain a more accurate image.

The image processing unit 140 may generate narrow band images, for example, a blue narrow band image, a green narrow band image, and a near infrared narrow image for each wavelength band based on the compensated image signals. The image processing unit 140 may combine the narrow band images of different wavelength bands into a single image in order to obtain images with an improved lesion contrast. For example, the image processing unit 140 may select any two images of the blue narrow band image, green narrow band image, and near infrared narrow band image and fuse them. As another example, the image processing unit 140 may fuse all of the narrow band images to generate a new narrow band image. Alternatively, the image processing unit 140 may fuse a color image obtained from white visible light and a blue narrow band image, a green band narrow image, or a near infrared narrow band image obtained from narrow band light to generate additional narrow images. The narrow band images or the fused additional narrow band images may be displayed on the display unit 150.

The endoscope apparatus 100 may use near infrared band light, and thus, may obtain images of portions of the cavity at a mucosal layer depth of about 200 to 300 μm and also at a sub-mucosal layer depth of about 500 to 1000 μm. In this respect, the endoscope apparatus 100 may obtain similar effects as those from an auto fluorescence imaging (AFI) endoscope or a chromoendoscope. Also, the endoscope apparatus 100 may obtain images of a plurality of wavelengths in a single capturing operation, thereby obtaining various narrow band images.

The illumination unit 110 may be configured based on a plurality of light sources. FIG. 6 illustrates another example of the illumination unit 110 of FIG. 1. Referring to FIG. 6, the illumination unit 110 may include a white light source 113W for emitting visible light of a white band, a blue light source 113B for emitting a narrow band blue light, a green light source 113G for emitting a narrow band green light, and a near infrared light source 113NIR for emitting a narrow band near infrared light. For example, a white light-emitting diode (LED) may be used as the white light source 113W. Also, a blue LED for emitting light of a band of about 380 to 450 nm may be used as the blue light source 113B, and a green LED for emitting light of a band of about 500 to 560 nm may be used as the green light source 113G, and an infrared LED for emitting light a band of about 800 to 860 nm may be used as the near infrared light source 113NIR. As another aspect, light sources such as a laser diode may also be used. It is understood that it is within the scope of the teachings herein that as long as light of desired wavelengths may be emitted, the type of the light sources is not limited, and any type of light source may be used.

In the above-described example of the illumination unit 110, in order to obtain a color image by illuminating in the cavity with white visible light (white light mode), the white light source 113W is turned on and the other light sources, namely the blue, green, and near infrared light sources 113B, 113G, and 113NIR, are turned off. To generate a narrow band image (narrow band mode), the white light source 113W is turned off, and the other light sources, namely the blue, green, and near infrared light sources 113B, 113G, and 113NIR, are turned on. The configuration of the sensing unit 130 and the image processing unit 140 may be similar to the sensing unit 130 and the image processing unit 140 of the example of FIG. 1.

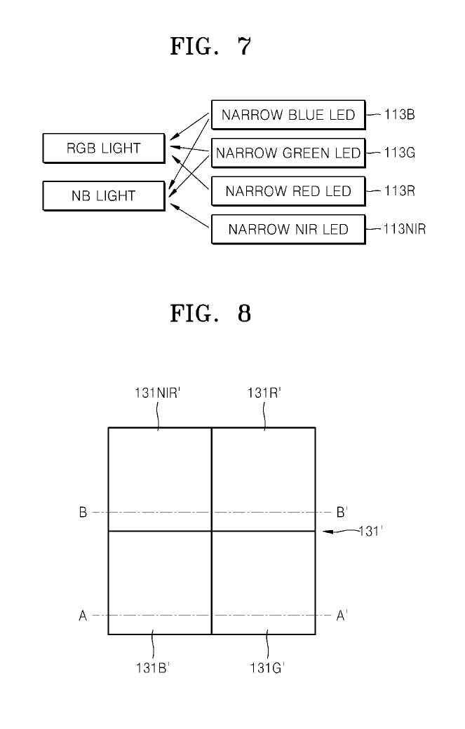

In addition, the illumination unit 110 may be formed of a plurality of narrow band light sources and omitting a white light source. FIG. 7 illustrates another example of the illumination unit 110 of FIG. 1. Referring to FIG. 7, the illumination unit 110 may include a blue light source 113B for emitting narrow band blue light, a red light source 113R for emitting narrow band red light, a green light source 113G for emitting narrow band green light, and a near infrared light source 113NIR for emitting narrow band near infrared light. The type of light sources is not limited to the example described above, and other implementations are within the scope of the teachings herein. For example, a blue LED for emitting light of a band of about 380 to 450 nm may be used as the blue light source 113B, and a green LED for emitting light of a band of about 500 to 560 nm may be used as the green light source 113G, and a red LED for emitting light of a band of about 600 to 660 nm may be used as the red light source 113R, and an infrared LED for emitting light of a band of about 800 to about 860 nm may be used as the near infrared light source 113NIR.

In the above-described example of the illumination unit 110, to obtain a color image by illuminating in the cavity with white visible light (white light mode), the blue light source 113B, the green light source 113G, and the red light source 113R are turned on, and the near infrared light source 113NIR is turned off. To generate a narrow band image (narrow band mode), the red light source 113R is turned off, and the other light sources, namely the blue, green, and near infrared light sources 113B, 113G, and 113NIR, are turned on. Also in this case, the configurations of the sensing unit 130 and the image processing unit 140 may be substantially the same as the sensing unit 130 and the image processing unit 140 of the example of FIG. 1.

In the case of the photodetection pixel 131 of the sensing unit 130 described above with reference to FIGS. 4, 5A, and 5B, the IR cut-off filter 13 is disposed on the red, green, and blue sub-pixels 131R, 131G, and 131B. Thus, the near infrared sub-pixel 131NIR senses near infrared rays by the near infrared sub-pixel 131NIR while the red, green, and blue sub-pixels 131R, 131G, and 131B sense almost no near infrared rays. However, a near infrared ray may also be sensed by all the sub-pixels. For example, FIG. 8 is a view illustrating another example of a photodetection pixel 131′ of the sensing unit 130 illustrated in FIG. 1. Referring to FIG. 8, the photodetection pixel 131′ of the sensing unit 130 may include a near infrared sub-pixel 131NIR for sensing near infrared light, a red sub-pixel 131R′ for sensing red light, a blue sub-pixel 131B′ for sensing blue light, and a green sub-pixel 131G′ for sensing green light.

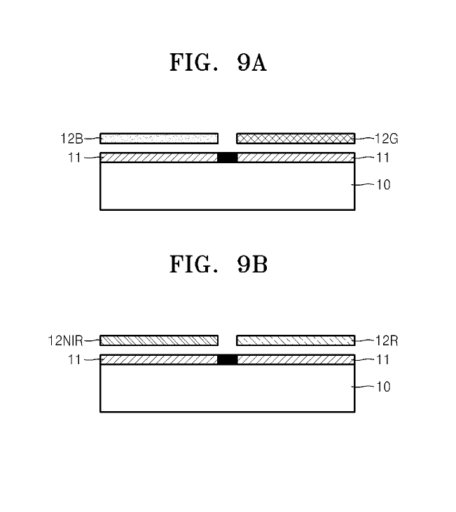

In this example, an IR cut-off filter is not disposed on the red sub-pixel 131R′, the blue sub-pixel 131B′, and the green sub-pixel 131G′. For example, referring to FIG. 9A, which is a cross-sectional view illustrating the example of the photodetection pixel 131′ illustrated in FIG. 8 taken along a line A-A′, a plurality of photosensitive layers 11 including photosensitive devices and wirings are disposed on a substrate 10, and a blue color filter 12B and a green color filter 12G are disposed on corresponding photosensitive layers 11. Also, referring to FIG. 9B, which is a cross-sectional view illustrating the example of the photodetection pixel 131′ illustrated in FIG. 8 taken along a line B-B′, the photosensitive layers 11 are disposed on the substrate 10, and a near infrared filter 12NIR and a red color filter 12R are disposed on corresponding photosensitive layers 11.

In response to an IR cut-off filter not being used as described above, a near infrared ray may be partially sensed by the red sub-pixel 131R′, the blue sub-pixel 131B′, and the green sub-pixel 131G′, and thus, an amount of the near infrared ray of the light reflected in the inside of the cavitycavity may be measured more accurately. As another aspect, in this case, red, blue, and green color information measured at the red sub-pixel 131R′, the blue sub-pixel 131B′, and the green sub-pixel 131G′, respectively, may be distorted. As another aspect, the red, blue, and green color information may be corrected based on the amount of the near infrared ray of the light measured at the near infrared sub-pixel 131NIR. For example, the image processing unit 140 may have values that are measured in advance with respect to ratios of sensing a near infrared ray by each of the red sub-pixel 131R′, the blue sub-pixel 131B′, and the green sub-pixel 131G′ with respect to the near infrared sub-pixel 131NIR. Thus, the image processing unit 140 may calculate a contribution ratio of the near infrared rays among the amount of the light measured at each of the red, blue, and green sub-pixels 131R′, 131B′, and 131G′ based on the values that are measured in advance. Accordingly, by subtracting each near infrared ray contribution amount from the amount of the light measured at each of the red, blue, and green sub-pixels 131R′, 131B′, and 131G′, accurate red, blue, and green color information may be obtained.

The sensing unit 130 illustrated in FIG. 8 may be combined with any of the light source units 110 illustrated in FIGS. 2, 6, and 7. Also, the image processing unit 140 may operate the same as described above. As another aspect, the image processing unit 140 may further calculate a contribution amount of a near infrared ray among the light amount measured from each of the red, blue, and green sub-pixels 131R′, 131B′, and 131G′, and accurately correct red, blue, and green color information.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. An endoscope apparatus comprising:
an illumination unit configured to selectively provide light of different wavelength bands and white light;
a sensing unit configured to generate an image signal by receiving light of a near infrared wavelength band and light of a visible light band; and
an image processing unit configured to generate a color image and a plurality of narrow band images from image signals of different wavelength bands generated by the sensing unit,
wherein the sensing unit comprises a plurality of photodetection pixels, each photodetection pixel comprising an infrared sub-pixel for sensing near infrared light, a red sub-pixel for sensing red light, a blue sub-pixel for sensing blue light, and a green sub-pixel for sensing green light.

2. The endoscope apparatus of claim 1, wherein the illumination unit comprises:
a white light source for emitting light of a near infrared ray band and white light of a visible light band; and
a filter member comprising a visible light filter that allows visible light to pass through and a narrow band filter that allows light of a plurality of different wavelength bands to pass through.

3. The endoscope apparatus of claim 2, wherein the narrow band filter allows blue light, green light, and near infrared light to pass through, and light that is emitted from the white light source and passes through the narrow band filter becomes light in which blue, green, and near infrared light are mixed.

4. The endoscope apparatus of claim 2, wherein the filter member is disposed on a light path in front of the white light source and is moved or rotated such that light emitted from the white light source is selectively passed through the visible light filter or the narrow band filter.

5. The endoscope apparatus of claim 1, wherein the illumination unit comprises a white light source for emitting white visible light of a wide band, a blue light source for emitting narrow band blue light, a green light source for emitting narrow band green light, and a near infrared ray light source for emitting narrow band near infrared light.

6. The endoscope apparatus of claim 5, wherein the endoscope apparatus operates in a white light mode that provides white visible light to a cavity or a narrow band mode that provides light of a plurality of narrow bands to the cavity, wherein in the white light mode, the white light source is turned on and the blue, green, and near infrared light sources are turned off, and in the narrow band mode, the white light source is turned off, and the blue, green, and near infrared light sources are turned on.

7. The endoscope apparatus of claim 1, wherein the illumination unit comprises a blue light source for emitting narrow band blue light, a green light source for emitting narrow band green light, a red light source for emitting narrow band red light, and a near infrared ray light source for emitting narrow band near infrared light.

8. The endoscope apparatus of claim 7, wherein the endoscope apparatus operates in a white light mode that provides white visible light to a cavity or a narrow band mode that provides light of a plurality of narrow bands to the cavity, wherein in the white light mode, the blue, green, and red light sources are turned on and the near infrared light source is turned off, and in the narrow band mode, the red light source is turned off, and the blue, green, and near infrared light sources are turned on.

9. The endoscope apparatus of claim 1, wherein the endoscope apparatus operates in a white light mode that provides white visible light to a cavity or a narrow band mode that provides light of a plurality of narrow bands to a cavity, and in the white light mode, the image processing unit generates a color image based on image signals transmitted from the sensing unit.

10. The endoscope apparatus of claim 9, wherein in the narrow band mode, the image processing unit generates a narrow band blue image, a narrow band green image, a narrow band near infrared image based on the image signals transmitted from the sensing unit.

11. The endoscope apparatus of claim 10, wherein the image processing unit generates additional narrow band images by fusing any two of or all of the blue, green, and near infrared narrow band images or fusing a color image obtained in the white light mode and one of the blue, green, and near infrared narrow band images obtained in the narrow band mode.

12. The endoscope apparatus of claim 1, wherein the image processing unit compensates for discoloration and a decrease in resolution generated in the sensing unit by using an interpolation method.

13. The endoscope apparatus of claim 1, wherein each of the photodetection pixels comprises:
a substrate;
a plurality of photosensitive layers arranged on the substrate; and
a blue color filter, a green color filter, a red color filter, and a near infrared ray filter disposed on corresponding photosensitive layers.

14. The endoscope apparatus of claim 13, wherein the image processing unit comprises values that are measured with respect to ratios at which near infrared rays are sensed at the red sub-pixel, the blue sub-pixel, and the green sub-pixel with respect to the near infrared sub-pixel.

15. The endoscope apparatus of claim 14, wherein the image processing unit calculates a contribution ratio of the near infrared rays among the amount of light measured at each of the red, blue, and green sub-pixels based on the values measured in advance, and corrects red, blue, and green color information in the red, blue, and green sub-pixels based on a calculation result.

16. The endoscope apparatus of claim 13, wherein each of the photodetection pixels further comprises an infrared ray cut-off filter that is disposed on the blue color filter, the green color filter, and the red color filter.

17. A method of obtaining an image of an endoscope apparatus, the method comprising:
illuminating a cavity by selectively providing light of a plurality of narrow bands of different wavelength bands including a near infrared band and white visible light to the cavity;
generating an image signal with respect to the plurality of different wavelength bands by receiving light reflected by the cavity;
generating a color image and a plurality of narrow band images from image signals with respect to the plurality of different wavelength bands; and
generating additional narrow band images by fusing the color image and the plurality of narrow band images.

18. The method of claim 17, where the illuminating is performed in a white light mode in which white visible light is provided to the cavity and a narrow band mode in which light of a plurality of narrow band wavelengths is provided to the cavity.

19. The method of claim 18, wherein the light of a plurality of narrow band wavelengths comprises narrow band blue light, narrow band green light, and narrow band near infrared light.

20. The method of claim 19, wherein the generating a color image and a plurality of narrow band images comprises generating a narrow band blue image, a narrow band green image, and a narrow band near infrared image based on image signals with respect to the narrow band blue light, the narrow band green light, and the narrow band near infrared light.

21. The method of claim 20, wherein the generating a color image and a plurality of narrow band images comprises generating a color image based on image signals with respect to the white visible light.

22. The method of claim 21, wherein the generating additional narrow band images comprises generating additional narrow band images by fusing any two narrow band images among a narrow band blue image, a narrow band green image, and a narrow band near infrared image or all the narrow band images or fusing the color image with the narrow band blue image, the green band narrow image, and the narrow band near infrared image.

23. The method of claim 17, wherein the generating an image signal comprises compensating for discoloration and a decrease in a resolution in a sensing unit that receives light reflected by the cavity, by using an interpolation method.

24. The method of claim 23, wherein the sensing unit comprises a plurality of photodetection pixels, each photodetection pixel comprising an infrared sub-pixel for sensing near infrared light, a red sub-pixel for sensing red light, a blue sub-pixel for sensing blue light, and a green sub-pixel for sensing green light.

25. The method of claim 24, wherein the generating an image signal comprises:
calculating a contribution ratio of the near infrared rays among the light amount measured at each of the red, blue, and green sub-pixels based on values measured with respect to ratios at which near infrared rays are sensed by the red sub-pixel, the blue sub-pixel, and the green sub-pixel with respect to the near infrared sub-pixel; and
correcting red, blue, and green color information of the red, blue, and green sub-pixels based on a calculation result.

26. The endoscope apparatus of claim 2, wherein the visible light filter filters light other than the visible light.

27. The endoscope apparatus of claim 2, wherein the narrow band filter filters light other than the blue, green and near infrared bands.

28. An endoscope apparatus comprising:
an illumination unit configured to emit light including red, green and blue bands and near infrared band;
a sensing unit configured to receive the light via at least one photodetection pixel, each including one sub-pixel corresponding to each one of the bands of the light; and
an image processing unit configured to generate an image based on the received light.
